(19) United States
(12) Patent Application Publication
(10) Pub. No.: US 2003/0100089 A1
(43) Pub. Date: May 29, 2003

(54) Serine protease inhibitor and processes for the preparation thereof

(76) Inventors: Eric Dupont, Ile d'Orleans (CA); Richard Beliveau, Ile-des-Soeurs (CA); Denis Gingras, Outremont (CA); Alain Renaud, Chambord (CA); France Cadoret, Coteau-Du-Lac (CA); Violetta Dimitriadou, Cap Rouge (CA); Pierre Falardeau, Sillery (CA)

Correspondence Address: HAYNES AND BOONE, LLP, 901 MAIN STREET, SUITE 3100, DALLAS, TX, 75202, US

(21) Appl. No.: 10/228,830

(22) Filed: Aug. 27, 2002

Related U.S. Application Data
(60) Provisional application No. 60/315,112, filed on Aug. 27, 2001.

(51) Int. Cl. C12N 9/99; C07K 16/40

(52) U.S. Cl. 435/184; 530/388.26; 530/395

(57) ABSTRACT

A composition comprising a protein having an inhibitory activity for a serine protease and, in particular, for a serine elastase, the protein having a molecular weight of about 46 kDa in a deglycosylated form, and having a molecular weight of about 54 kDa when isolated in a glycosylated form is provided. Methods of using the protein in treatments of diseases where inhibition of a serine protease and, in particular, of a serine elastase is effective are provided as well as compositions, reagents, and kits related thereto.

### FIELD OF THE INVENTION

The present invention relates generally to a glycoprotein having an apparent molecular weight of about 54 kDa (p54), which has serine protease inhibitor activity (a serpin-like molecule). The invention also relates to a process for preparing the same, methods as well as compositions for treating, preventing or alleviating the symptoms of disorders and diseases associated with an excess level of serine protease. Amongst these diseases are psoriasis, emphysema, pulmonary hypertension, liver fibrosis, anaemia, diseases characterized by tumor growth or invasion, as well as any disease involving mast cells.

### BACKGROUND OF THE INVENTION

Cartilage extracts and processes for obtaining them are disclosed in International Patent Publication Nos. WO 95/32722, WO 96/23512 and WO 97/16197. Inhibitor activities of proteinases have been searched and found in cartilage extracts obtained from sharks. Namely, an anti-collagenolytic activity is known to be present in shark cartilage extract.

Serine protease inhibitors or “serpins” form a large family of inhibitors that regulate various physiological processes such as angiogenesis, neoplasia, apoptosis, inflammation, coagulation, fibrinolysis and viral pathogenesis. Dysfunction of such serine protease inhibitors has been implicated in cancer, thrombosis, emphysema, cystic fibrosis, liver cirrhosis, psoriasis, arthritis, immune hypersensitivity, and mental disorders (See Yamaguchi K, et al., A potential novel peptidase, resembling but distinct from neutrophil elastase, produced by carcinoma cells, Oncol. Rep. 7:1017-21, 2000; Janciauskiene S, Conformational properties of serine proteinase inhibitors (serpins) confer multiple pathophysiological roles, Biochim. Biophys. Acta 1535:221-235, 2001; Maas N, et al., Maspin-a novel protease inhibitor with tumor-suppressing activity in breast cancer, Acta. Oncol. 39:931-934, 2000).

Alpha 1-antitrypsin (AAT), an acute-phase reactant during inflammation response, has a physiological role in controlling tissue destruction by endogenous serine proteases. AAT is a glycoprotein having a molecular weight of 53 kDa which is primarily synthesized in the liver, and to a lesser extent by macrophages and neutrophils (Coakley R J, al., Alpha 1-antitrypsin deficiency: biological answers to clinical questions, Am. J. Med. Sci. 321:33-41, 2001). AAT is the most common physiological inhibitor of various elastases including neutrophil elastase, cathepsin G and proteinase 3. It has been recently approved to specifically treat emphysema under the orphan drug act in the United States (Biotech Patent News July 2001: pp 4-5).

Prolastin™ (Bayer corp.) is indeed currently used to treat congenital alpha 1-anti trypsin deficiency which is associated with slowly developing emphysema. This inhibitor is prepared from pooled human plasma of normal donors with all the precautions required to remove the potential viral infectivity. Because there is no totally effective decontamination known for the removal of viral infectivity, any safer alternative source of products than blood borne products may be desirable. Prolastin™ is suspended to a concentration of about 20 mg/ml. It is given at a rate of 0.08 ml/kg/minute or greater, intravenously. A recommended dosage of 60 mg/kg takes approximately 30 minutes to infuse.

Alpha 1-antichymotrypsin is a serine protease inhibitor synthesized in the liver with an apparent affinity toward chymotrypsin-like enzymes. Alpha 1-antichymotrypsin is structurally related to AAT. It is a single glycopeptide chain of approximately 68 kDa (Eriksson S G, et al, Serine proteinase inhibitors as acute phase reactants in liver disease, Tokai. J. Exp. Clin. Med. 13:365-71, 1988). Alpha I-antichymotrypsin may be useful in the treatment of pulmonary inflammation (U.S. Pat. No. 4,916,117).

Elastase is a serine proteinase, that is able to break down mainly elastin and also connective tissue proteins such as fibronectin, collagen and cartilage tissues (Reilly C, et al., The degradation of human lung elastin by neutrophil proteinases, Biochem. Biophys. Acta, 621:147-167 1980; Mainardi C L, et al., Degradation of type IV (basement membranes) collagen by a proteinase isolation from human PMN leucocyte granules, J. Biol. Chem. 255: 5436-5441 1980). It can be particularly problematic when its activity is unregulated, or when AAT is not present in sufficient quantities to regulate the activity of elastases. AAT deficiency is in fact, a common lethal hereditary disorder. Clinical signs of AAT deficiency are often observed in the lungs or the liver showing a high risk of emphysema and liver disease. The degradation of elastin associated with emphysema probably results from a local imbalance of elastinolytic enzymes and the naturally occurring tissue and plasma proteinase inhibitors. AAT replenishment has been successfully used for treatment of such disorders (Campbell E J, et al., Quantum proteolysis by neutrophils: implications for pulmonary emphysema in alpha 1-antitrypsin deficiency, J. Clin. Invest. 104:337-44, 1999). AAT has also been approved for the treatment of individuals with congenital panacinar emphysema.

Serine elastase inhibitors are also useful in the treatment of primary pulmonary hypertension, since disease progression is associated with increased serine elastase activity (Cowan K N, et al., Complete reversal of fatal pulmonary hypertension in rats by a serine elastase inhibitor, Nat. Med. 6:698-702, 2000).

Neutrophils are a source of serine elastase and cathepsin G which contribute to tissue damage of inflammatory diseases, especially cystic fibrosis, thus indicating that a serine elastase inhibitor may be useful in the treatment of such disorders.

A cartilage-derived leucocyte protease inhibitor isolated from bovine cartilage has been described in U.S. Pat. No. 4,746,729. This inhibitor has a molecular weight of about 15 kDa, and an isoelectric point greater than 9.5. The diseases to be treated with this inhibitor comprise the inflammatory diseases, including pulmonary diseases such as emphysema, chronic bronchitis, cystic fibrosis, bronchiectasis, and adult respiratory distress syndrome. Other inflammatory diseases include atherosclerosis, arthritis, psoriasis, vasculitis, glomerulonephritis, consumption coagulopathies associated with gram-negative sepsis, and leukemias.

U.S. Pat. No. 4,243,582 discloses the purification from bovine cartilage of two glycoproteins of about 65 kDa. These proteins have an inhibitory activity against trypsin and endothelial cell growth, and an isoelectric point of about 3.8.

U.S. Pat. No. 4,845,076 describes a protease inhibitor called HUSI (human seminal plasma inhibitor) Type I, which includes similar inhibitors isolated from other tissues, named CUSI-I (cervix-uterus-secretion-inhibitor) and BSI (bronchial-secretion-inhibitor). HUSI-I and CUSI-I have a molecular weight of about 11 kDa, are acid resistant and have anti-elastase activity. BSI has a molecular weight of about 10 kDa, cross-reacts with anti-HUSI-I antibodies, is acid resistant and inhibits HLE, cathepsin-G, trypsin and chymotrypsin. European Patent Application No. 346,500 discloses that SLPI (which is secretory leukocyte protease inhibitor, and the same as HUSI-I) also is an elastase inhibitor, namely a HLE inhibitor. The C-terminal portion of SLPI appears to be an elastase binding domain.

U.S. Pat. No. 5,290,762 discloses the use of protease inhibitors having affinity for mast cell mediators, plasma kinins or T-cell mediators as anti-inflammatory agents. The target mediators comprise cathepsin-G and elastase. One of the inhibitors is alpha-2-antiplasmin which has 11% carbohydrate content, comprises asparagine and leucine as the amino terminal residues, and a molecular weight of about 65 to 70 kDa.

U.S. Pat. No. 5,618,786 discloses aerosols comprising alpha₁-antitrypsin for treating emphysema. Elastase, which is a target for alpha₁-antitrypsin is said to be implicated as a major cause of this disease. In addition to alpha₁-antitrypsin, aerosols may comprise other proteins that appear to be determinant in the resorption of respiratory diseases, such as interferons, immunoglobulins, lipocortin, phospholipase inhibitors and atrial natriuretic factor. All these proteins are known to affect hallmarks of inflammation and/or edema.

International Patent Publication No. WO96/08275 discloses the use of SLPI or HUSI-I as an inhibitor of tryptase, another serine protease which is found in large quantities in mast cells. Apparently, this enzyme is responsible for the degradation of vaso-intestinal peptide (VIP), a broncho-relaxant peptide.

U.S. Pat. No. 4,760,130 discloses a serine protease inhibitor which, like SLPI, has an elastase-binding domain and a trypsin-binding domain. The inhibitor has a molecular weight of about 12 kDa and is obtained from parotid secretions. The profile of affinity of this inhibitor toward different serine proteases shows a much greater affinity for HLE and chymotrypsin than for trypsin (a ten-fold difference) and than for cathepsin-G and PE (a one hundred-fold difference).

U.S. Patent Publication No. 2001/0006939 discloses compositions comprising SLPI in the form of a dry powder that is highly dispersible in a gas. Such aerosols are intended to be used to treat inflammatory pulmonary diseases.

A human monocyte elastase inhibitor called HEI has been cloned and described in U.S. Pat. No. 5,827,672. The cytosolic protein has a molecular weight of about 42 kDa, is stable to reducing agents, is non-glycosylated and forms a covalent complex with elastases, namely the porcine pancreatic elastase, a human neutrophil elastase. It is possible that a glycosylated form of HEI exists in the extracellular environment.

Although many protease inhibitors are known, they have a specific profile of affinity for given proteases. There is a need for inhibitors having different specificity or selectivity for given proteases, in order to provide therapeutic agents to be used alone or in combination with other pharmaceutical compounds.

### SUMMARY OF THE INVENTION

The present invention is directed to a novel inhibitor of serine proteases. The inhibitor is a glycoprotein having a molecular weight of about 54 kDa (hereinafter referred to as “p54 protein” or “p54”). The p54 glycoprotein has a protein backbone of about 46 kDa (hereinafter referred to as “p46 protein” or “p46”). The p46 protein shows the same activity against serine proteases as p54. The p54 protein is isolated from shark cartilage. It is assumed that species-variants can be obtained from other sources of cartilage and that variants could be obtained synthetically or by mutagenic protocols to introduce substitutions, additions or deletions which would provide a protein that is still functional insofar as inhibition of serine proteases is concerned. Variants would be called “p54-like” or “p46-like” proteins. A process for obtaining the p54 or p46 proteins, as well as the use of the p54 or p46 proteins in compositions for the treatment or prevention of diseases involving proteases, namely elastases (such as cancer, neovascularization and inflammation) in animals, are within the scope of this invention.

From another aspect, the present invention provides methods for treating diseases associated with an excess level of serine proteases, or elastase activity, rheumatoid arthritis, emphysema, pulmonary hypertension, psoriasis, liver fibrosis and diseases characterized by tumor growth and invasion.

The present invention also relates to a method and composition for treating mammals afflicted with a disease involving pulmonary mast cells. More particularly, the present invention relates to the direct or prophylactic treatment of certain diseases involving pulmonary mast cells, by administering the present protease inhibitor, its analogs, salts, derivatives, nucleic acid derivatives or DNA vector expressing the p54 gene or p54 DNA sequences derived from p54, alone or in combination with anti-inflammatory agents (e.g., corticosteroids). When combined with corticosteroids, a synergistic effect is observed. More particularly, a synergistic effect is observed when such combinations are provided by inhalation therapy for treating asthma or related sinusitis.

Mast cells have been found to be implicated in diseases and events such as allergic and non-allergic rhinitis, nasal polyposis, atopic dermatitis, psoriasis, contact dermatitis, pancreatitis, emphysema, asthma, colitis, Crohn's Disease, wound healing, cluster headaches, coronary artery spasm and rheumatoid arthritis.

According to another embodiment, the present invention provides for antibodies directed specifically against p54 or p46 and methods for detecting p54 or p46 by using these specific antibodies.

According to yet another embodiment, the present invention provides methods of purification of p54 or p46 using an affinity medium comprising antibodies directly specifically against p54 or p46.

The following embodiments and figures are part of the present specification and are included to further demonstrate certain aspects of the invention. The invention may be better understood by reference to one or more of these figures in combination with the detailed description of the specific embodiments presented herein, which do not have the purpose of limiting the scope of the invention.

### BRIEF DESCRIPTION OF THE DRAWINGS

FIG. 1 depicts a molecular profile of cartilage extract separated with a gel-filtration column. Fractions showing elastase—inhibiting activity are shown by dotted lines.

FIG. 2 depicts a separation profile of the cartilage protein p54 kDA polypeptide with an anion exchange column and a correlation with p54 on an SDS polyacrylamide electrophoresis gel.

FIG. 3 depicts the inhibitory effect of the p54 on porcine pancreas elastase (PPE) and human leukocyte elastase (HLE).

FIG. 4 depicts the molecular weight of unglycosylated p54.

FIG. 5A and FIG. 5B depict the survival rate (FIG. 5A) and pulmonary arterial pressure (FIG. 5B) as affected by the oral administration of a composition containing the p54 polypeptide in an experimental lung hypertension rat model.

### DETAILED DESCRIPTION OF THE INVENTION

The serine protease inhibitor of the present invention is obtainable from shark cartilage. The serine protease inhibitor of the present invention may be obtained according to the following process:

1) Obtaining a 0-500 kDa fraction: The 0-500 fraction is a shark cartilage liquid extract comprising components having molecular weights less than about 500 kDa. Preparative methods for the 0-500 fraction are disclosed in the International Publication Nos. WO 95/32722, WO 96/23512, and WO 97/16197, the entire disclosures of which are hereby incorporated by reference. The disclosed methods comprise the steps of:

a) homogenizing shark cartilage in an aqueous solution in conditions compatible with the preservation of the integrity of biologically active components present in cartilage until the cartilage is reduced to solid particles whose size is less than about 500 μm;

b) extracting said biologically active components into the aqueous solution, which results in a mixture of solid particles and of crude liquid extract (LE) having the biologically active components;

c) separating said liquid extract from the solid particles;

d) further separating the crude liquid extract so as to obtain a final liquid extract containing molecules having molecular weights less than about 500 kDa (LE-0-500); and

e) filtering the LE-0-500 on a microfiltration membrane (0.22 micron) and freezing to obtain the final liquid extract (0-500 fraction).

2) Obtaining a 0-1 kDa fraction and a 1-500 kDa fraction: The 0-1 fraction is a shark cartilage liquid extract comprising components having molecular weights less than about 1 kDa. The 1-500 fraction is a shark cartilage liquid extract comprising components having molecular weights between about 1-500 kDa. Preparative methods for the 0-1 and 1-500 fractions are disclosed in International Publication Nos. WO 95/32722, WO 96/23512, and WO 97/16197. The disclosed methods comprise the steps of:

f) filtering the LE-0-500 with a membrane having a nominal molecular weight cut-off of about 1 kDa to form permeate liquid extracts comprising cartilage molecules having molecular weights less than about 1 kDa (P 0-1), and retentate liquid extracts (R 0-1) comprising cartilage molecules having molecular weights greater than about 1 kDa; and

g) microfiltering the retentate and permeate liquid extracts through a microfiltration membrane having a porosity of about 0.22 microns.

The 1-500 fraction was then concentrated, applied on a gel-filtration column and fractions showing PPE-inhibiting activity were pooled. Subsequently, the active fractions were applied on an affinity-column, washed and eluted, and then active fractions were pooled. Determination of the molecular weight of this active fraction revealed the presence of three major bands, one of which shows PPE inhibiting activity.

The active protein has an apparent molecular weight of 54 kDa and as noted above is referred to herein as “p54”. Partial amino acid sequences of p54 revealed that it is a novel protein since no substantial overall amino acid sequence homology with any known proteins has been found.

Determination of the molecular structure of p54 revealed that it is a glycoprotein, the core protein moiety of which has an apparent molecular weight of 46 kDa. Moreover, p46 was surprisingly found to be active against PPE.

It was found that p54 and p46 are non-competitive inhibitors of elastases. P54 was also found to inhibit other types of proteolytic enzymes such as chymotrypsin, trypsin and plasmin. It was not found to significantly inhibit matrix metalloproteinases (MMP), thrombin, cathepsins and pepsin. Overall, p54 or p46 are serine-protease inhibitors that also show selectivity for elastases.

Evaluation of the stability of p54 indicated that this inhibitory protein is quite stable. Such stability will facilitate mass production of p54 with a negligible loss of activity. Loss of activity may result from denaturation occurring during the processes of mass production, storage, and transport. Furthermore, p54 is quite abundant in the liquid cartilage fraction, thus facilitating the recovery of large quantities of the product.

The p54 protein of the present invention can be applied to the treatment of diseases associated with an excess level of elastase activity, such as cancer, rheumatoid arthritis, emphysema, lung hypertension, liver fibrosis, and psoriasis.

Conversely, an excess of serpin may also be the subject of a pathology and of a treatment. Several biological events or pathologies appear to involve overexpression of serpin molecules. The overexpression of AAT appears to inhibit the activity of a protease called PACE-4, which leads to abnormal embryogenic development (Tsuji et al., Inactivation of proprotein convertase, PACE4, by alpha 1-antitrypsin Portland (alpha 1-PDX), a blocker of proteolytic activation of bone morphogenetic protein during embryogenesis: evidence that PACE4 is able to form an SDS-stable acyl intermediate with alpha 1-PDX, J Biochem. (Tokyo) 1999 September;126(3): 591-603). Neutralizing AAT could also be useful in preventing an IgE synthesis (Jeannin, P. et al., Alpha-1 antitrypsin up-regulates human B cell differentiation selectively into IgE- and IgG4- secreting cells, Eur J Immunol. 1998 June;26(8):1815-22). AAT further appears to be a mediator of altered iron metabolism, characteristic of anaemia (Graziadei, I. et al., Unidirectional upregulation of the synthesis of the major iron proteins, transferrin-receptor and ferritin, in HepG2 cells by the acute-phase protein alpha 1-antitrypsin, J Hepatol 1997 October;27(4):716-25). When overexpressed, AAT also reversed the effect of another protein convertase called PC7 which is involved in the maturation of β-amyloid precursor protein. Any means by which AAT would be decreased or neutralized could lead to the control of the progression of Alzheimer's disease (Lopez-Perez et al., Proprotein convertase activity contributes to the processing of the Alzheimer's beta-amyloid procursor protein in human cells: evidence for a role of the prohormone convertase PC7 in the constitutive alpha-secretase pathway, J Neurochem 1999 November;73(5):2056-62). Therefore, anti-serpin molecules such as anti-AAT antibodies, inhibitors, antagonists or antisense oligo- or poly- nucleotides could find a clinical use as anti-serpin-like molecules, and anti-p54 and p46 antibodies or anti-molecules could also find therapeutic applications.

Antibodies specific for p54 and p46 were produced by inoculation of an appropriate animal with each of these polypeptides. Anti-p54 antibodies are specific for p54 and they bind to the glycosylated form. Anti-p46 antibodies are specific p46 and they bind to the non-glycosylated form (p46). Antibody production includes not only the stimulation of an immune response by injection into animals, but also analogous steps in the production of synthetic antibodies or other specific-binding molecules such as the screening of recombinant immunoglobulin libraries, the utilization of transgenic animals or the in vitro stimulation of lymphocyte populations (Winter, G. and Milstein, C., Man-made antibodies, Nature 349:293-299, 1991). Those of ordinary skill in the art will recognize that these techniques may be adapted to produce molecules that specifically bind to p54 and p46 as well as to isolate analogues of these polypeptides. These antibodies may also be suitable for administration in an animal to target or inhibit p54 and p46 homologues (envisaged doses would be equivalent to an intravenous dose of about 1 mg to about 10 mg per Kg of body weight) as well as to quantify the presence of p54 or p46 in any biological fluid. This could be useful to evaluate the pharmacokinetic parameters of any p54 or p46 treated animals or humans.

The p54 or p46 proteins may be administered in a suitable therapeutic dose to animals and humans as a bioactive agent. Further, DNA vectors may be used to express p54 or p54 derivatives in the animal body. Additionally, the bioactive agent may be complexed with a variety of well established compounds or compositions which enhance stability or pharmacological properties such as half-life. It is evident in the art that the therapeutic, bioactive composition may be delivered by intravenous infusion or any other effective means or routes which could be used for treating problems involving excess expression and activity of proteases, namely elastase.

The scope of the present invention is further illustrated but not limited in the following examples.

### EXAMPLE 1

### Purification of the p54

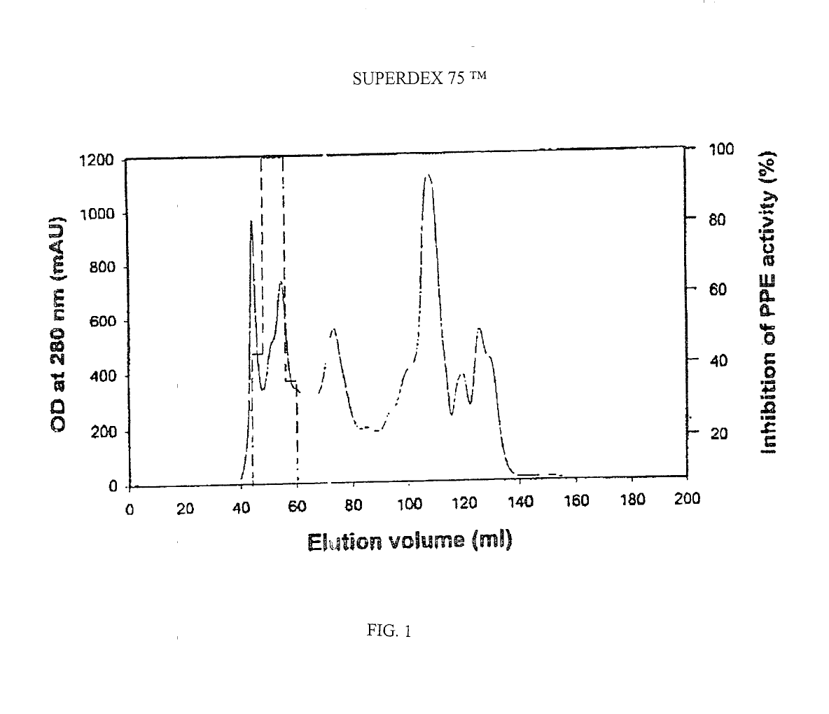

The concentrated 1-500 fraction was applied on a SUPERDEX 75™ gel-filtration column (Pharmacia, Baie d'Urfé, Québec) which was pre-equilibrated with Tris-HCl (Buffer A: 20 mM, pH 7.4). The flow rate was 0.5 ml/min. The profile chromatogram of protein concentrations was monitored at a 280 nm wavelength and the optical density was represented as arbitrary units (mUA). Fractions showing elastase-inhibiting activity were pooled (shown by dotted lines in FIG. 1).

The pooled fraction was pre-cleared on a Heparin-Sepharose column. Briefly, sepharose beads were incubated with active fraction for 1 hour at 4° C. The beads were spun and the supernatant was then incubated for 3 hours at 4° C. with beads cross-linked to lectin-glycine (Glycine Max gel; Sigma, St-Louis) which retains glycosilic chains containing N-acetyl-L-galactosamine (Sigma). The supernatant containing the Glycine max gel was then mounted on a column to permit sedimentation of the gel, and the flow through solution was discarded. The column was then washed with buffer A followed by buffer A containing 100mM NaCl, before an elution with buffer A containing 100 mM N-acetyl-D-galactosamine.

The eluted fraction was then applied on a 9% SDS-Polyacrylamide gel electrophoresis PAGE (120 volt). Coomassie Blue staining of the polyacrymalide gel indicated the presence of a protein band having a molecular weight of 54 kDa.

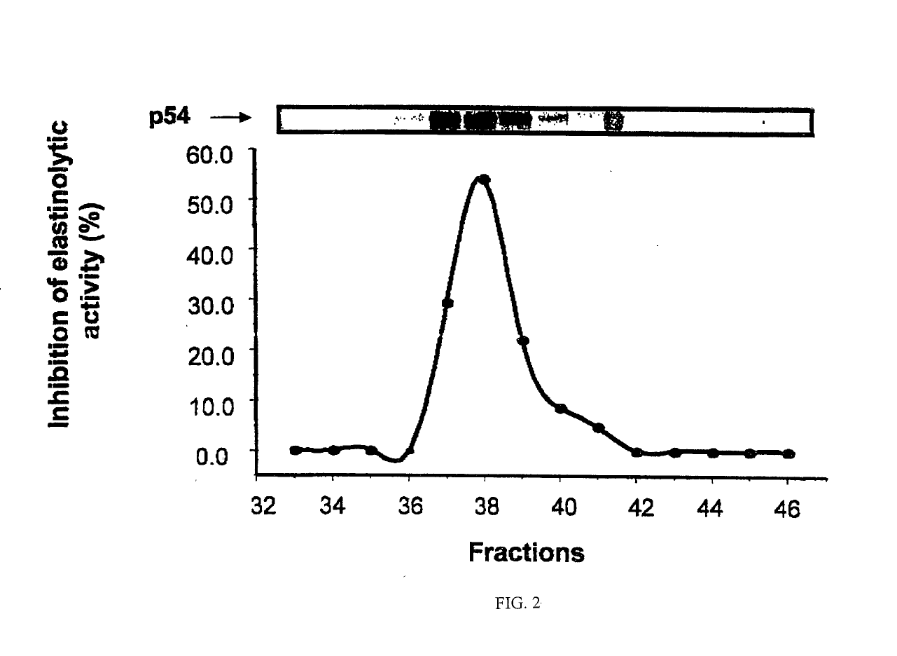

The samples were then applied to an anion exchange column MONOQ²HR 5/5™ (Pharmacia) previously equilibrated with 20 mM Tris-HCl (pH 10). Elution was performed at 1 ml/min using an exponential NaCl gradient and 0.5 ml fractions were collected. Elastase activity in these fractions was monitored using a commercial kit as indicated below (FIG. 2). The presence of the 54 kDa molecular weight protein was monitored by SDS polyacrylamide electrophoresis (FIG. 2).

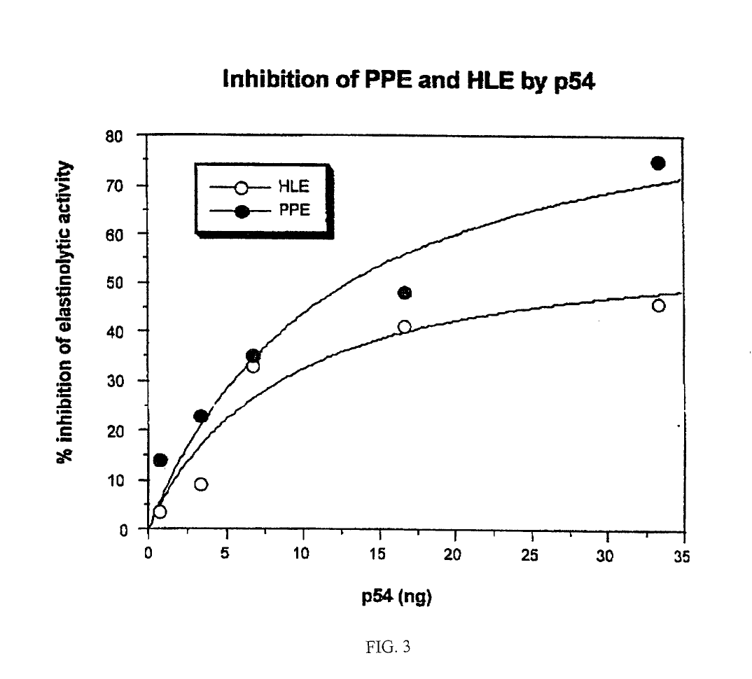

The inhibitory activity of these fractions was monitored using an elastase assay with a porcine pancreatic elastase (PPE). The PPE assay was performed using a commercial kit (Molecular probes, Oregon). It is used to determine the ability of test samples to inhibit elastase activity. Briefly, the assay was performed as follows. An elastin substrate (elastin labeled with a fluorescent conjugate: BODIPY FL) was loaded in a microplate with PPE in the absence or in the presence of the test sample. The elastin substrate is cleaved by PPE to release a fluorescent peptide. PPE activity was estimated by the amount of fluorescent peptide present in the incubation mixture. Fluorescence was determined using a microplate reader (excitation 485 nm, emission 530 mm). High PPE activity thereby yields high signals, and a low PPE activity in turn (e.g. by addition of an inhibitor) causes low signals. The inhibition of elastase by the p54 protein was also validated with a human leukocyte elastase (FIG. 3).

Steps for purification of p54 are summarized in Table I. Protein was quantitatively assayed by the bicinchoninic acid method (Smith et al., Measurement of protein using bicinchoninic acid. Anal. Biochem. 150:76-85, 1985:) the entire disclosure of which is hereby incorporated herein by reference. One unit (1 U.) was defined as 1% of inhibition.

TABLE I

| | Total protein | Specific Activity | Enrichment | Yield |
|---|---|---|---|---|
| Step | (mg) | (Units/mg) | (fold) | (%) |
| 1-500 | 127.6 | 12,866 | 1.0 | 100.0 |
| S-75 | 9.44 | 50,319 | 3.9 | 28.9 |
| G-Max | 0.06 | 1,121,500 | 87.2 | 4.1 |
| MonoQ | 0.0075 | 1,804,067 | 140.2 | 0.8 |

The amount of p54 found in the liquid cartilage extract was estimated as about 0.1% (w/w), which is quite abundant.

### EXAMPLE 2

### Determination of Amino Acid Sequence of the p54

The partial amino acid sequence of p54 prepared in Example 1 was determined by N-terminal sequence Edman degradation employing an amino acid sequence analyzer. Comparison of amino acid sequences between the N-terminal sequence of the p54 and other proteins, including known elastase-inhibiting proteins, confirmed that the p54 protein of the present invention is a novel protein. Eleven other internal sequences were obtained from fragments of p54 that were obtained by digestion with trypsin. From these sequences, three revealed a 55% to 66% homology with MT of different species. SEQ ID NO: 6 revealed 70% homology with human serpin B12. This sequence was obtained by hybrid quadrupole time-of-flight mass spectroscopy (Q-TOF; Kristensen et al., Mass spectrometric approaches for the characterization of proteins on a hybrid quadrupole time-of-flight (Q-TOF) mass spectrometer. Electrophoresis 21:430-9, 2000).

| | |
|---|---|
| Partial amino acid sequence of p54: | |
| N-terminal sequence: | |
| • KPVAPNENIPSQL (Y/L)ALK(T)AA | SEQ ID No:1 |
| Internal sequences: | |
| • PVAPNENIP | SEQ ID No:2 |
| • LAAANTEFALR | SEQ ID No:3 |
| • XHYEIQEXX | SEQ ID No:4 |
| • FSDDAN(L/I)SG(L/I)SE: | SEQ ID No:5 |
| 66% homology with human ATT | |
| • FFSP(L/I)S(L/I)SAA(L/I)SM(L/I)SQR: | SEQ ID No:6 |
| 70% homology with human SerpinB12 | |
| • ESYT(L/I)NE(L/l)(L/I)K: no homology | SEQ ID No:7 |
| • (SE or TD) EA(L/I)NVNFK: no homol- | SEQ ID No:8 |
| ogy | |
| • Q(L/I)AAQPSGTAK: no homology | SEQ ID No:9 |
| • EQ(L/I)NTYVSK: no homology | SEQ ID No:10 |
| • YEADXTTTVNVQFMTK: 55% homology with | SEQ ID No:11 |
| mouse AAT | |
| • N(L/I)SG(L/I)SENDP(L/I)QVSK: 64% | SEQ ID No:12 |
| homology with squirrel AAT | |

X stands for an unidentified amino acid
(L/I) means that I and L cannot be differentiated
One-letter codes for amino acids are as follows:

| | |
|---|---|
| Alanyl | A |
| Arginyl | R |
| Asparagyl | N |
| Aspartyl | D |
| Cysteyl | C |
| Glutaminyl | Q |
| Glutamyl | E |
| Glycyl | G |
| Histidyl | H |
| Isoleucyl | I |
| Leucyl | L |
| Lysyl | K |
| Methionyl | M |
| Phenylalanyl | F |
| Prolyl | P |
| Seryl | S |
| Threonyl | T |
| Tryptophyl | W |
| Tyrosyl | Y |
| Valyl | V |

### EXAMPLE 3

### P54 is a Glycoprotein

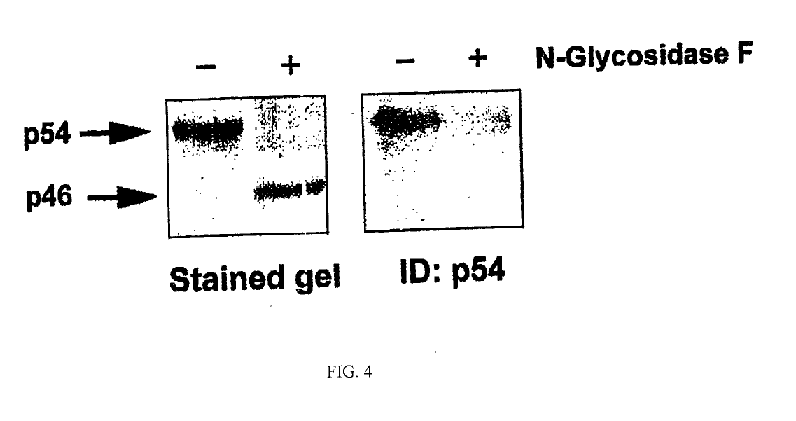

Incubation of p54 with the glycolytic enzyme, N-glycosidase F (Roche Molecular Biochemicals, Laval, Québec), which cleaves N-linked oligosaccharides generates N-deglycosylated a polypeptide with an apparent molecular weight of 46 kDa (p46) thus indicating that p54 is a glycoprotein and that p46 may correspond to the proteinaceous backbone of p54. (FIG. 4). Moreover, p46 may be useful since it shows elastase activity equivalent to that of p54. This also means that production of the present inhibitor would be possible in cellular systems, notwithstanding their capacity to glycosylate proteins.

### EXAMPLE 4

### Production of Specific Antibody Against the p54 and p46 Proteins

Antibodies specific for p54 (Ab-anti-p54) were produced by inoculation of a rabbit with the polypeptide using standard protocols. Briefly, 5 to 10 μg of the polypeptide were injected with the Complete Freund adjuvant (Pierce Co, Rockland, Ill.). Six weeks later, the polypeptide with incomplete Freund Adjuvant were injected to boost the animal. Sera of immunized animals were collected two weeks later by cardiac punction. Ab-anti-p54 does not recognize p46, thus indicating that the Ab-anti-p54 targets the glycosyl moiety of p54, and/or the physical structure of the binding site (epitope) for the Ab-p54 is modified in p46. An antibody specific for p46 was also produced (Ab-p46) by inoculating rabbits with p46. Monoclonal specific antibodies as well as recombinant immunoglobulins may also be produced using standard procedures as previously described (Harlow and Lane (1988) Antibodies: A Laboratory Manual. Cold Spring Harbor Laboratory, Cold Spring Harbor N.Y.; Goding (1986) Monoclonal Antibodies: Principles and Practice, Academic Press, New York City). More specifically, the protocols used for production of monoclonal antibodies against different domains of α₁-anti trypsin (AAT) could be reproduced with p54 or p46, which antibodies can further be used in the manufacture of diagnostic reagents and kits, and for the design of assays for detecting serpin-like molecules. Such antibodies, assays and kits for AAT are disclosed in Herion, P. et al., Monoclonal antibodies against plasma protease inhibitors: II. Production and characterization of 25 monoclonal antibodies against human alpha 1-antitrypsin. Correlation between antigenic structure and functional sites, Biosci Rep 1984 February; 4(2):139-147; Wallmark, A. et al., Monoclonal antibody specific for the mutant PiZ alpha 1-antitrypsin and its application in an ELISA procedure for identification of PiZ gene carriers, Proc Natl Acad Sci USA 1984 September 81(18):5690-3; Zhu, X J and S K Chan, The use of monoclonal antibodies to distinguish several chemically modified forms of human alpha 1-proteinase inhibitor, Biochem J 1987 August 15;246(1):19-23; Zhu, X J et al., The identification of epitipic sites in human alpha 1-proteinase inhibitor, Biochem J 1987 August 15;246(1):25-36; Silvestrini, B. et al., Development of an enzyme-linked immunosorbent assay with a monoclonal antibody prepared against alpha 1-antitrypsin for diagnostic screening of inflammatory disorders, Clin Chem 1990 February;36(2):277-82; Kramer, M D et al., Measurement of free human leukocyte elastase and human leukocyte elastase/alpha 1 proteinase inhibitor complexes by an enzyme-linked immunosorbent assay, J Immunol Methods 1990 July 20;131(1):41-8; Trefz, G. et al., Establishment of an enzyme-linked immuno-sorbent assay for urinary trypsin inhibitor by using a monoclonal antibody, J Immunoassay 1991;12(3):347-69; Abbink, J. J. et al, Production of monoclonal antibodies against inactivated alpha 1-antitrypsin. Cross-reactivity with complexed alpha 1-antitrypsin and application in an assay to determine inactivated and complexed alpha 1-antitrypsin in biological fluids, J Immunol Methods 1991 October 25;143(2):197-208), which contents are all incorporated by reference. Antibodies against another serpin molecule, a human monocyte elastase inhibitor (HEI), are also disclosed in U.S. Pat. No. 5,827,672, the contents of which are herein incorporated by reference.

### EXAMPLE 5

### Tests Using p54 and p46 Specific Antibodies

Specific antibodies may be useful for the pharmacokinetic analysis of these polypeptides. Diagnostic tests for p54 and p46 include methods utilizing the antibody and a label to detect p54 and p46 in animal body fluids, tissues or extracts of such tissues. The polypeptides and antibodies of the present invention may be used with or without modification since they are frequently labeled by joining them, either covalently or noncovalently, with a substance that provides a detectable signal. A wide variety of labels and conjugation techniques have been reported extensively. Suitable labels include, but are not limited to, radionuclides, enzymes, substrates, cofactors, fluorescent and chemiluminescent agents, label-containing agents and magnetic particles. Protocols for measuring p46 and p54 using specific antibodies are techniques known in the art. Examples include, but are not limited to, enzyme-linked immunosorbent assay (ELISA), radioimmunoassay (RIA) and fluorescent activated cell sorting (FACS).

The antibodies may also be useful in a process of isolation or purification wherein a retention medium, namely an affinity column comprising the same would be used to retain p54 or p46. A new process could be redesigned to take into account the new affinity-based purification.

The antibodies may also be used as inhibitors for p54 or p46 (and therefore as an activator for the serine-proteases) when they bind to regions important or critical for the recognition and inhibition of the target serine-proteases). The antibodies may finally be used as a mean for targeting a cell, tissue or organ to be treated or to eliminated. The antibodies could be conjugated directly to toxic moieties, such as drugs, chemicals, radiotoxic agents or indirectly to lipid vesicles such as liposomes, the vesicles comprising these agents.

### EXAMPLE 6

### Specificity of P54

The activity of p54 towards other types of proteases in addition to elastases (PPE and HLE) revealed that p54 also inhibits to a lesser extent other serine proteinases such as chymotrypsin (53%), plasmin (49%) and trypsin (30%), whereas it does not inhibit MMP-2 and MMP-7, cathepsin D, cathepsin G, thrombin and papain (see table III). In contrast, AAT inhibits all the serine proteinases tested; chymotrypsin (100%), trypsin (94%), PPE (64%), Plasmin (49%), and cathepsin G (68%) and the aspartate proteinase cathepsin D (77%). These findings indicate that the action of p54 is more specific for serine elastases, a subclass of serine proteinases, than AAT.

TABLE III

| | | Activity (% inhibition) | |
|---|---|---|---|
| Enzyme | Class of protease | p54 | AAT |
| MMP-2 | Metallo proteinase | 0 | N/D |
| MMP-7 | Metallo proteinase | 0 | 0 |
| Cathepsin G | Serine proteinase | 0 | 68 |
| PPE | Serine proteinase | 84 | 64 |
| HLE | Serine proteinase | 42 | ND |
| Thrombin | Serine proteinase | 0 | ND |
| Chymotrypsin | Serine proteinase | 53 | 100 |
| Trypsin | Serine proteinase | 30 | 94 |
| Plasmin | Serine proteinase | 49 | 49 |
| Papaine | Cysteine proteinase | 8 | ND |
| Cathepsin D | Aspartate proteinase | 0 | 77 |

### EXAMPLE 7

### Nucleic Acids

Nucleic acids encoding a protein which comprises any one of SEQ ID NO's: 1 to 12 are under the scope of this invention. Indeed, any nucleic acid having at least 12 nucleotides, preferably at least 17, and more preferably at least 27 nucleotides in length, which is capable of specifically hybridizing under stringent or non-stringent conditions with the nucleic acids encoding p54 or p46 is within the scope of this invention. Nucleic acids may be designed, synthesized based upon the amino acid sequences 1 to 12, taking into account the codon degeneracy, and used as probes to find longer sequences for different tissues and species. Once more complete definite sequences of nucleic acids obtained from a plurality of given species and tissues are obtained, variance of the sequences is visualized by aligning the same. Conserved regions can lead to the design of probes or primers hybridizable under stringent conditions (see Maniatis et al. Molecular Biology: A Laboratory Manual, Cold Spring Harbor Laboratory, Cold Spring Harbor, N.Y.) to the gene sequence of a plurality of species. Less conserved regions may lead to the design of probes or primers suitable for hybridization under less or non stringent conditions to reveal gene sequences from a variety of species, or for hybridization under stringent conditions to one or a limited set of closely related species. The nature and the length of a probe or a primer is selected upon its G+C content, its capacity to avoid forming secondary and tertiary structures and its specificity for a complementary target sequence. Nucleic acids can be used as probes to detect serpin-like molecules such as p54 or p46 genes (tissue, cell, lysate, whole body) of one or a variety of species. Variants are intended to cover nucleic acids (and proteins) which, despite some sequence changes have substantially the same function and activity profile be it a species variant, a naturally occurring variant or an artificially created mutant. Nucleic acids can also be used as primers to amplify and/or detect serpin-like genes or gene transcripts. Nucleic acids are valuable complements or alternatives to antibodies in diagnostic reagents, kits and assays, as well as in therapeutic applications (to increase or to silence the expression of p54 or p46). Nucleic acids may also be used for manufacturing the proteins p54, p46 or congeners in other species, or polypeptides, or peptides of interest, by recombinant technology. The nucleic acids therefore cover recombinant vectors and especially expression vectors, that are to be used in a compatible host cell to produce the proteins or the peptides under basal or induced conditions. A large panoply of expression vectors and of gene promoters can be used to govern the expression of p54 or p54-like genes. A strategy for obtaining the gene sequences and the total deduced amino acid sequence of p54 or p46 can be derived from the teachings of U.S. Pat. No. 5,827,672, the contents of which are incorporated herein by reference.

Nucleic acids may also be used for gene therapy. When used in a “sense” orientation, these nucleic acids would express or overexpress the gene product. When used in an “antisense” orientation, the nucleic acids would silence the expression of the p54 gene.

### EXAMPLE 8

### Biological Activity of p54

Pulmonary hypertension (PAH) is commonly associated with congenital heart defects, pulmonary diseases associated with chronic hypoxia, hepatic disorders and connective tissue disease. It is a vascular disease which affects the blood vessels between the heart and lungs known as the pulmonary blood vessels. Blockage of blood flow through the circulatory system causes elevated pulmonary blood pressure, increasing the strain on the right side of the heart as it tries to pump blood to the lungs.

A cause and effect relationship has been shown between endogenous vascular elastase (EVE) and experimentally induced pulmonary hypertension in experimental animal models (Zhu, et al., The endogenous vascular elastase that governs development and progression of monocrotaline-induced pulmonary hypertension in rats is a novel enzyme related to the serine proteinase adipsin, J. Clin. Invest., 94: 919-920 (1994); Cowan et al., Complete reversal of fatal pulmonary hypertension in rats by a serine elastase inhibitor Nature Medicine 6: 698-702, 2000).

Increased pulmonary artery elastinolytic activity associated with the monocrotaline-induced pulmonary hypertension model has been shown to be moderated by treatment with an elastase inhibitor (Ye, et al., Inhibition of elastolysis by SC-37698 reduces development and progression of monocrotaline pulmonary hypertension, Am. J. Physiol. 261 (Heart Circ. Physiol. 30): H1255-H1267 (1991); Cowan et al., Elafin, a serine elastase inhibitor, attenuates post-cardiac transplant coronary arteriopathy and reduces myocardial necrosis in rabbits afer heterotopic cardiac transplantation, J. Clin Invest., 97:2452-2468 (1996); Cowan et al., Complete reversal of fatal pulmonary hypertension in rats by a serine elastase inhibitor Nature Medicine 6: 698-702, 2000). In some models, early inhibition of EVE activity largely prevented pulmonary vascular damage. Although, EVE has been shown to be sensitive to leukocyte elastase (LE) inhibitors, it is believed that it is a novel enzyme distinct from LE. Inhibitors of EVE may be useful in treating pulmonary vascular disease in infants, restenosis secondary to angioplasty, pulmonary hypertension myocarditis, bronchopulmonary dysplasia, myocardial necrosis after cardiac transplant, post-cardiac transplant coronary arteriopathy, atherosclerosis and reperfusion injury following myocardial infarct.

Adult male Sprague-Dawley rats (250-300 g in body weight; Charles River Laboratories, St-Constant, QC, Canada) were subcutaneously injected with 60 mg/kg monocrotaline (Sigma) to induce pulmonary hypertension. In addition to a group of untreated rats, the experimental group included rats that received daily gavage tube feedings with 2.5 ml of the composition containing p54 at a dose of about 1.25 μg of p54 per day (which makes a dose of p54 of about 3 to 5 μg per Kg of body weight per day as an enteral dose for rats).

Survival analysis and hemodialysis were performed as previously described by Cowan (Cowan et al., Complete reversal of fatal pulmonary hypertension in rats by a serine elastase inhibitor Nature Medicine 6: 698-702, 2000). Briefly, animals were euthanized according to criteria determined and set forth by the Animal Care Committee. These endpoints included a sustained bradycardia of less than 100 beats/min, arterial oxygen tension of less than 80%, and abrupt weight loss with a reduction in body weight of more than 10% per day for 2 days. Rats were evaluated for these criteria by the veterinary staff of our Animal Facility, who recommended killing without knowledge of the treatment group. Direct pulmonary artery catheterization was done and pressures were measured in anesthetized rats.

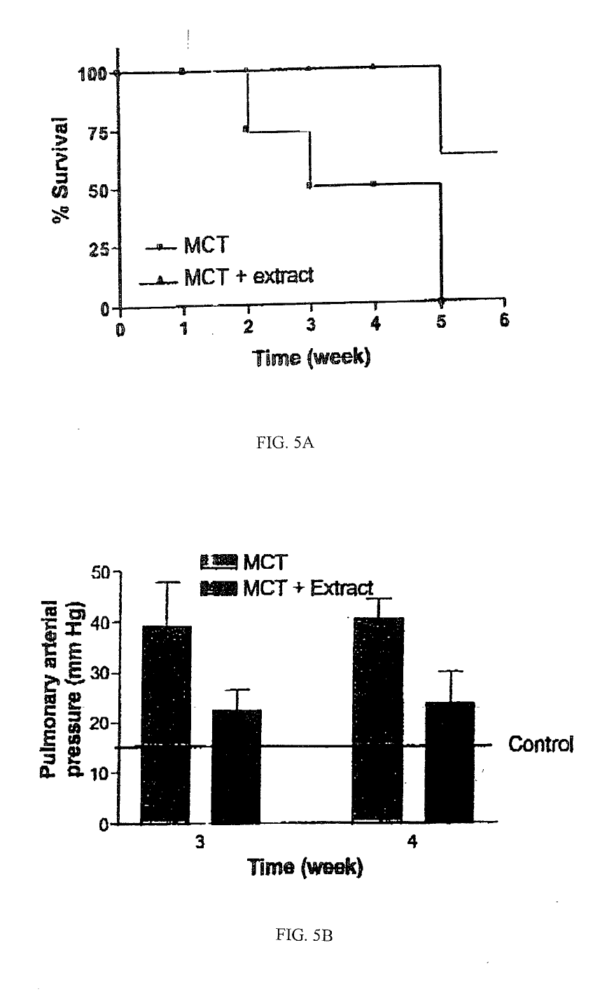

The effect of a composition of cartilage extract containing p54 was evaluated on the monocrotaline-induced pulmonary hypertension model. It was shown that such extract increases rat survival and reduces pulmonary arterial pressure of monocrotaline-treated rats (see FIG. 5A and FIG. 5B) thus indicating that such composition containing p54 could be useful in the treatment of pulmonary hypertension. The dose of a composition comprising p54 could be increased to achieve a p54 dose of 2 to 10 fold the dose of 1.25 μg given to the rats.

An inhibitor related to p54 such as α₁-anti trypsin is also implicated in the reduction of pulmonary hypertension and elastase inhibitors comprising α-keto heterocycles also moderated the same (U.S. Pat. No. 6,159,938). This supports the findings that p54 is at least in part responsible for the effects observed. Prostacyclin levels appear to be a marker monitorable and correlated with an improved lung tension, and such levels could be used along with the survival rate and the pressure to evaluate the efficiency of variants. It is presumed that p54 could advantageously replace AAT for treating diseases involving enzymes targeted by this type of inhibitors.

### EXAMPLE 9

### Medical uses and Formulations

The present invention also relates to a method and composition for treating mammals afflicted with pulmonary mast cell-implicating disease. More particularly, the present invention relates to the direct or prophylactic treatment of such diseases, by administering serine protease inhibitors, their analogs, salts or derivatives, alone or in combination with other anti-inflammatory agents such as corticosteroids whereby a synergistic or additive effect is found. Preferably, the inhibitors comprise p54 or p46. According to the present invention, there is particularly provided compositions for treating asthma by inhalation therapy and for related sinusitis.

Mast cells have been found to be implicated in diseases and events such as allergic and non-allergic rhinitis, nasal polyposis, atopic dermatitis, psoriasis, contact dermatitis, pancreatitis, emphysema, asthma, colitis, Crohn's Disease, wound healing, cluster headaches, coronary artery spasm and rheumatoid arthritis. Mast cells produce proteases and an inhibitor such as the p54 protein of the present invention is believed to counteract the effect of mast cells and thus to treat a mast cell-implicating disease.

The above results provide credible evidence that the inhibitor of the present invention can be used to treat inflammatory diseases, particularly those involving mast cells. A dosage regimen ranging from 0.01-1000 μg/Kg/day, preferably from 1-10 μg/Kg/day is envisaged. Molecules of the same class as p54 have been systematically used or are intended to be used to treat inflammatory diseases, namely respiratory inflammatory diseases as well as other diseases such as cancer, pulmonary hypertension and post-operative hemorrhages, due to a variety of mediators or cell events that include kinines and hyperfibrinolysis. There is a strong likelihood that p54 could be used in the same manner as molecules of the same class such as alpha₁-antitrypsin and SLPI.

U.S. Pat. No. 5,290,762, the entire disclosure of which is hereby incorporated herein by reference, discloses anti-inflammatory topical formulations which comprise 0.5-2% of protease inhibitors. A solution of 20% inhibitors for the treatment of colitis and administered as an enema is also disclosed. It is contemplated and within the scope of the present invention to prepare similar formulations with similar inhibitor percentages for the making of pharmaceutical formulations comprising p54 or p46.

U.S. Pat. No. 5,618,786, the entire disclosure of which is hereby incorporated herein by reference, discloses aerosols which may comprise 0.1-5% of anti-inflammatory proteins. Alpha₁-antitrypsin for example is administered in a dose achieving one microgram to ten milligrams per kilogram of host body weight, at a frequency of one or more times per day. Such formulations normally comprise lipid particles having a size of about 0.5-5 micrometers. It is contemplated and within the scope of the present invention to prepare similar formulations and doses of p54 or p46 for the making of therapeutic formulations.

International Patent Publication WO96/08275, the entire disclosure of which is hereby incorporated herein by reference, discloses SLPI-comprising formulations that are of different types: depots for subcutaneous or intra-muscular administration, aerosols, osmotic pumps or devices, which are all disclosed to show the possibility of making fast release and slow release compositions of inhibitors. It is contemplated and within the scope of the present invention to prepare similar formulations by substituting at least a part of SLBI with p54 or p46.

A composition comprising SLBI in the form of a dry powder is disclosed in U.S. Patent Publication 2001/0006939, the entire disclosure of which is hereby incorporated herein by reference. A composition comprising 10% SLBI by weight of water, which powder itself comprises 50-95% particles of 1-8 micrometers is disclosed. This composition is dispersible in a gas and provides for a high level of dispersability. SLBI is used in an aerosol form comprising the dry powder in the milligram range (2-20 mg) per day. Such types of compositions are also within the scope of the present invention. Such compositions would be prepared by substituting at least a part of SLBI with p54 or p46.

A composition comprising p54 or p46 may be complemented with another anti-inflammatory-type agent. Examples of such complementary agents include a cartilage extract (an extract “spiked” with p54 or p46), cortico-steroids, kinins inhibitors or antagonists, prostaglandins inhibitors or antagonists, etc.

Although the present invention has been described hereinabove by way of preferred embodiments thereof, it can be modified, without departing from the spirit and nature of the subject invention as defined in the appended claims.

What is claimed is:

1. A protein having an inhibitory activity towards elastinolytic serine proteases, which is either:
a glycosylated protein having an apparent molecular weight of about 54 kDa, and having a proteinaceous backbone of an apparent molecular weight of about 46 Kda, wherein said protein is isolable from cartilage, or a partially or totally deglycosylated form thereof.

2. A protein according to claim 1, wherein said protein is glycosylated and has an apparent molecular weight of about 54 kDa.

3. A protein according to claim 1, wherein said protein is deglycosylated and has an apparent molecular weight of about 46 kDa.

4. A protein according to claim 1, which is obtained from shark cartilage and which comprises any one of the amino acid sequences of SEQ. ID NO's: 1-12.

5. An antibody directed against the protein of claim 2.

6. An antibody directed against the protein of claim 3.

7. A nucleic acid encoding any one of the amino acid sequences defined in SEQ. ID NO's: 1-12.

8. A nucleic acid of at least 12 nucleotides in length hybridizing with a nucleic acid as defined in claim 7.

9. A nucleic acid of at least 17 nucleotides in length hybridizing with a nucleic acid as defined in claim 7.

10. A nucleic acid of at least 27 nucleotides in length hybridizing with a nucleic acid as defined in claim 7.

11. A method for detecting the presence of an anti-elastase serpin in a sample, a tissue or a cell, which comprises the steps of:
contacting a sample, tissue or cell with the antibody of claim 5 in conditions supporting the formation of a bound complex between said serpin and antibody, and
detecting the presence of said bound complex as an indication of the presence of said serpin in said sample, tissue or cell.

12. A method for detecting the presence of an anti-elastase serpin in a sample, a tissue or a cell, which comprises the steps of:
contacting a sample, tissue or cell with the antibody of claim 6 in conditions supporting the formation of a bound complex between said serpin and antibody, and
detecting the presence of said bound complex as an indication of the presence of said serpin in said sample, tissue or cell.

13. A diagnostic reagent comprising the antibody of claim 5.

14. A diagnostic reagent comprising the antibody of claim 6.

15. A diagnostic kit, which comprises a protein as defined in claim 1 and an antibody directed against the protein.

16. A diagnostic kit, which comprises a protein as defined in claim 2 and an antibody directed against the protein.

17. A method for detecting the expression of an anti-elastase serpin-like gene in a sample, a tissue or a cell, which comprises the steps of:
contacting the nucleic acids obtained from a sample, tissue or cell with a nucleic acid as defined in claim 7 in conditions supporting the formation of a hybridization complex, and
detecting the presence of said hybridization complex as an indication of the expression of said serpin-like gene in said sample, tissue or cell.

18. The method of claim 17 further comprising a step of amplification of at least a portion of said nucleic acids.

19. A diagnostic reagent comprising a nucleic acid as defined in claim 8.

20. A diagnostic kit which comprises at least one nucleic acid as defined in claim 7.

21. A diagnostic kit which comprises at least two nucleic acid as defined in claim 7, capable of priming amplification of at least a portion of said nucleic acid.

22. A method for treating a disease which involves an elastinolytic serine protease, which comprises the step of administering an effective amount of a protein as defined in claim 1 to a subject in need of such a treatment.

23. A method as defined in claim 22, which is an inflammatory disease.

24. A method as defined in claim 22 wherein said disease is selected from psoriasis, emphysema, pulmonary hypertension, liver fibrosis, diseases characterized by tumor growth or invasion, arthritis, thrombosis, cystic fibrosis, cirrhosis, immune hypersensitivity, chronic bronchitis, atherosclerosis, vasculitis, rhinitis, nasal polyposis, dermatitis, colitis, pancreatis, coronary artery spasms, cluster headaches, wound healing and asthma.

25. A composition for treating a disease which involves an elastinolytic serine protease, which comprises an effective amount of a protein as defined in claim 1, and a pharmaceutically acceptable vehicle.

26. A composition as defined in claim 25, which further comprises an anti-inflammatory agent.

27. A composition as defined in claim 26, wherein said anti-inflammatory agent is a corticosteroid, a cartilage extract, a kinin synthesis inhibitor, a kinin receptor antagonist, a prostaglandin synthesis inhibitor or antagonist.

28. A process for obtaining an inhibitor of a serine elastase, which comprises the steps of:
obtaining a mixture of an aqueous solution and of cartilage particles of a size of about 500 μm;
extracting soluble components from cartilage into said aqueous solution, thereby obtaining an extract comprising said inhibitor;
separating said extract from said cartilage particles;
fractionating said extract on a gel filtration medium;
monitoring an activity against a serine elastase in each fraction; and
recovering a fraction or a plurality of adjacent fractions having said activity.

29. A process as defined in claim 28, wherein said cartilage is shark cartilage.

30. A process as defined in claim 29, wherein said fraction or fractions comprise an inhibitor having a molecular weight of about 54 kDa in a glycosylated form or of about 46 kDa in a deglycosylated form.

31. A process as defined in claim 30, wherein, prior to the step of fractionating on a gel filtration medium, a step of fractionating on a filter membrane of a molecular weight cut-off value of 500 kDa and a step of recovering an extract having molecules of a molecular weight lower than 500 kDa, are performed.

32. The use of a cartilage extract which is either the fraction comprising molecules of a molecular weight lower than 500 kDa or the fraction obtained upon gel filtration, as defined in claim 31 for inhibiting elastinolytic serine proteases, which comprises the step of contacting a sample, a cell or a tissue with said cartilage extract.


SEQUENCE LISTING (12 sequences)

SEQ ID NO: 1 - protein, 20 aa, Squalus sp.
  MISC_FEATURE (14)..(14): Xaa = Tyrosyl or Leucyl
KPVAPNENIPSQLXALKTAA

SEQ ID NO: 2 - protein, 9 aa, Squalus sp.
PVAPNENIP

SEQ ID NO: 3 - protein, 11 aa, Squalus sp.
LAAANTEFALR

SEQ ID NO: 4 - protein, 9 aa, Squalus sp.
  MISC_FEATURE (1)..(9): Xaa = unidentified amino acid
XHYEIQEXX

SEQ ID NO: 5 - protein, 12 aa, Squalus sp.
  MISC_FEATURE (7)..(10): Xaa = Leucyl or Isoleucyl
FSDDANXSGXSE

SEQ ID NO: 6 - protein, 17 aa, Squalus sp.
  MISC_FEATURE (5)..(14): Xaa = Leucyl or Isoleucyl
FFSPXSXSAAXSMXSQR

SEQ ID NO: 7 - protein, 10 aa, Squalus sp.
  MISC_FEATURE (5)..(9): Xaa = Leucyl or Isoleucyl
ESYTXNEXXK

SEQ ID NO: 8 - protein, 10 aa, Squalus sp.
  MISC_FEATURE (1)..(1): Xaa = Seryl or Threonyl
XXEAXNVNFK

SEQ ID NO: 9 - protein, 11 aa, Squalus sp.
  MISC_FEATURE (2)..(2): Xaa = Leucyl or Isoleucyl
QXAAQPSGTAK

SEQ ID NO: 10 - protein, 9 aa, Squalus sp.
  MISC_FEATURE (3)..(3): Xaa = Leucyl or Isoleucyl
EQXNTYVSK

SEQ ID NO: 11 - protein, 16 aa, Squalus sp.
  MISC_FEATURE (5)..(5): Xaa = unidentified amino acid
YEADXTTTVNVQFMTK

SEQ ID NO: 12 - protein, 15 aa, Squalus sp.
  MISC_FEATURE (2)..(11): Xaa = Leucyl or Isoleucyl
NXSGXSENDPXQVSK